(19) United States
(12) Patent Application Publication
(10) Pub. No.: US 2015/0033430 A1
(43) Pub. Date: Feb. 5, 2015

(54) FACE PROTECTOR FOR WELDER

(71) Applicant: Optrel AG, Wattwil (CH)

(72) Inventors: Ramon Hofer Kraner, Herisau (CH); Daniel Iranyi, Steinhausen (CH); Leo Keller, Ruti (CH); Arno Lenzi, Zurich (CH); Tom Staubli, Zurich (CH); Uwe Werner, Hutten (CH)

(73) Assignee: Optrel AG, Wattwil (CH)

(21) Appl. No.: 14/021,020

(22) Filed: Sep. 9, 2013

(30) Foreign Application Priority Data
Aug. 5, 2013 (CH) 01353/13

(51) Int. Cl. A61F 9/06

(52) U.S. Cl. CPC A61F 9/06
USPC 2/8.2

(57) ABSTRACT

A face protector for protecting a welder's eyes and face includes an auto-darkening welding filter (ADF) unit that, in turn, includes a filter pane or ADF pane and an electronics unit driving the ADF pane. The electronics unit is configured to switch the ADF pane automatically from a transparent state to a darkened state when a welding operation takes place. A hard shell unit includes a shell carrying the ADF unit with the ADF and being shaped to protect a wearer's face, the shell being made from a plastic material A soft shell unit includes a cap for protecting the crown of the wearer's head and a neck protector for protecting the wearer's neck. The soft shell unit is made of flexible fabric with support elements.

### BACKGROUND OF THE INVENTION

1. Field of the Invention

The invention relates to the field of welding protection gear and in particular to a face protector for protecting a welder's eyes and face from glare and welding splatter.

2. Description of Related Art

U.S. Pat. No. 5,544,361 describes a lightweight headgear for protecting a wearer's face from ultraviolet radiation. It comprises a face shield with shield segments that, in a retracted position, nest with each other. The face shield can be attached to a baseball cap or to eyeglasses.

U.S. Pat. No. 4,965,887 describes a face protector for splash and splatter protection in the medical field. It comprises a bent rectangular plastic shield attached to a hood impermeable to fluids. It can be attached to a baseball cap or to eyeglasses.

U.S. Pat. No. 4,724,550 describes a cap and goggles combination comprising a rubber cap and a face glass to keep hair and face dry when swimming. The face glass is curved and comprises a cutout resting on the bridge of the wearer's nose.

EP 1789835 B1 describes an optically decentered face shield in which an optical centre of the face shield is displaced away from the normal straight ahead line of sight toward an activity specific line of sight, e.g. for one eye and for a particular sports activity.

U.S. Pat. No. 8,042,958 describes a welding mask with an automatic darkening filter with automatic power management. Power to the filter is turned on or off depending on movement of the device, detected by a motion sensor.

U.S. Pat. No. 7,945,297 describes an audio headset comprising a capacitive sensor to detect the presence of a user. Power consumption is reduced when a user is not present.

Welding protection equipment has made significant progress during the last couple of years. Active, opto-electronic auto-darkening welding filters allow hands-free operation and the uninterrupted control of the different welding processes. Auto-darkening welding filters (ADF) have gained the ability to automatically select the appropriate shade number according to the intensity changes of the welding arc, and, the switching speed from the transparent to the attenuated state has increased by a factor of 40. The protection of the eyes and the skin of the welder against optical radiation is represented by minimum requirements that today are fulfilled by almost every single product available. Accidents and frequent arc eyes are becoming the exception.

However, the basic concepts underlying anti-glaring protection helmets themselves have not changed much over the last hundred years: A very stiff and bulky face protector is mounted on a harness or on a headgear that is then fixed on the head by physical friction. The number of parts required and the necessity for flipping up the helmet create a framework in which a relatively heavy optical system, in most cases built as replaceable cartridge, leads to a tunnel-like viewing characteristic and adds weight, creating neck strain and fatigue. The result, in combination with the tensions created by the tight fit of the headgear on the head and the large distance of the face protector from the head, is discomfort, headaches and operational hindrance to do the welding job efficiently, especially in confined space environments.

### BRIEF SUMMARY OF THE INVENTION

It is an object of the invention to create a welding protection mask which allows for a larger field of view than commonly known welding masks.

It is a further object of the invention to create a welding protection mask which is light weight and is comfortable to carry, in particular by having an improved distribution of weight.

It is a further object of the invention to create a welding protection mask which is easy to put on and take off, increasing the efficiency of the welding process.

At least one of these objects is at least partially achieved by face protectors according to the patent claims.

The face protector comprises an auto-darkening welding filter (ADF) unit which in turn comprises a filter pane or ADF pane and an electronics unit driving the ADF pane, the electronics unit being configured to switch the ADF pane automatically from a transparent state to a darkened state when a welding operation takes place. The face protector further comprises a hard shell unit and a soft shell unit attached to each other. The hard shell unit comprises a shell carrying an ADF unit with the ADF and being shaped to protect a wearer's face. The shell is manufactured of a plastic material. The soft shell unit comprises a cap for protecting the crown of the wearer's head and a neck protector for protecting the wearer's neck, the soft shell unit being made of a combination of flexible fabric with support elements.

In this manner, soft and hard materials are combined, providing a face (and eye) protector or welding cap or welding face protector cap with increased comfort, as compared to conventional welding masks, and simplifying a fast and easy putting on and off functionality of the face protector. By using different material means, high structural stability and impact and process temperature resistance can be achieved with at the same time low cost manufacturing opportunities.

The face protector does not require a separate headgear for holding the protective parts. Rather, the elements for supporting the weight of the protective parts, when in use, are integrated in the soft shell unit and in particular in the cap.

The face protector can have one-size-fits-all capability. It allows close working distance to the weld (e.g. for shipyard applications) and supports confined space applications. Overall, the efficiency of a welder can be enhanced by these factors without compromising on safety, comfort, fatigue and long-term physical health.

The hard shell unit can be realised with different choices of material, depending on requirements:

high structural stability can be achieved by employing injection moulded flame retardant polyamide (PA) structures;

low cost manufacturability can be achieved by employing thermoformed flame retardant polycarbonate structures (PC); and

high resistance against impact and process temperature can be achieved by employing thermoformed epoxy materials which are reinforced by glass fibre materials.

The soft shell unit can be realised with different choices of material, depending on requirements:

low cost manufacturability can be achieved by employing inherently flame retardant fabric materials;

low cost manufacturability can be achieved by employing impregnated flame retardant cotton materials;

high resistance against process temperature can be achieved by employing flame retardant leather materials; and

high resistance against process temperature can be achieved by employing flame retardant polyimide materials (PI).

In certain embodiments, the face protector is configured such that when it is worn by a wearer, a distance (d) from the wearer's pupils to the ADF pane lies in the range of 30 mm to 50 mm, in particular in the range of 35 mm to 45 mm. In certain embodiments, a vertical extension h of the transparent part of the ADF pane is at least 40 mm, in particular at least 45 mm. In certain embodiments, a horizontal extension w of the transparent part of the ADF pane is at least 100 mm, in particular at least 110 mm. In certain embodiments, a lower edge of the ADF pane comprises a concave cutout for accommodating the wearer's nose.

Such measures, alone or in combination, allow to achieve on the one hand a close-to-the-eye configuration with a large field of view, compared to current products, and on the other hand a compact and low weight design.

Furthermore, since the front parts are lightweight and closer to the wearer's face, the distribution of the weight of the face protector can be better balanced than in conventional welding mask solutions. This reduces fatigue and neck strain and makes the face protector comfortable to carry.

In certain embodiments, the ADF pane is curved or spherical. For example, side portions of the ADF pane, relative to a central portion of the ADF pane, can curve backwards. That is, the side portions or lateral portions are curved in the direction of the wearer's face. By following the contour of the wearer's face the hard shell unit can be made even more compact and/or the field of view can be increased even more.

In certain embodiments, the hard shell unit comprises a protective shield, the protective shield being attached by means of a releasable connection, the protective shield optionally being shaped such that a plurality of protective shields can be stacked on each other, nesting with each other. Such protective shields are subject to wear and damage and can therefore be user replaceable.

In certain embodiments, the cap is shaped in the manner of a baseball cap, comprising a rounded crown and a stiff visor projecting in front, the visor covering a transverse top section of the shell, the top section protecting a forehead space enclosed by the hard shell unit, and the visor supporting at least some of the weight of the hard shell unit when the face protector is worn by a wearer. This gives the face protector on the one hand an appealing form and on the other hand allows to do away with a separate headgear for carrying the protective elements.

In certain embodiments, cap comprises a stiffener (or reinforcement or bracing) sewn in or attached to the other parts of the cap, the stiffener holding up the visor against at least part of the weight of the hard shell unit. The stiffener can be considered stiff as compared to the fabric parts of the cap. The stiffener can be an integrally formed piece of a plastic material, extending over the area of the visor and extending toward the back of the cap around at least part of the horizontal circumference of the cap. The stiffener can extend around at least half of the horizontal circumference of the cap, or at least to the region of the wearer's ears.

In certain embodiments, a top section, two side sections and a front section of the shell form a box-like structure that gives the shell structural stability, and wherein the front section comprises a convex section that is curved in a convex shape, and a nose bridge section that is shaped to extend at an angle to the convex section, further stiffening the front section.

In certain embodiments, the shell comprises lateral side sections, a lower section of each side section being shaped to keep the neck protector distanced from the wearer's neck, thereby establishing a ventilation space between the neck protector and the wearer's neck. The ventilation space can lead to a breathing space in front of the wearer's mouth and nose, and to a forehead space in front of the wearer's forehead.

In certain embodiments, the lower sections of the side sections are made of thinner material than the remaining sections of the side sections, and optionally also thinner than the other sections of the shell. This helps to reduce the overall weight of the mask.

In certain embodiments, the ADF pane is mounted on the shell by means of a frame section of the ADF frame, the frame section being attached to, in particular glued or welded to, the shell and holding the ADF pane in position between the frame section and the shell. This helps to reduce the overall weight of the mask since no separate frame or holder or cassette is used to hold the ADF pane or the entire ADF unit.

In certain embodiments, the electronics unit is mounted on the shell by means of a cover section of the ADF frame, the cover section being attached to, in particular glued or welded to, the shell and holding the electronics unit in position between the cover section and the shell. This also helps to reduce the overall weight of the mask, again because there is no separate housing for the electronics unit.

In certain embodiments, the frame section and the cover section are integrally shaped as a single piece. This simplifies production and assembly and makes connecting and sealing the ADF frame to the shell easier.

In certain embodiments, the ADF frame comprises at least one latch, the ADF frame is mounted at the inside of the shell with the latch reaching through a slot of the shell and forming a snap fit with the protective shield, holding the protective shield in place on the outer side of the shell. This further reduces the number of parts. It also makes for a better mechanical fit of the protective shield to the shell: If only the shell and the protective shield with their relatively stiff materials were involved, it would be more difficult or impossible to create a releasable snap action connection between the two.

In certain embodiments, at least the frame section and the latch are integrally shaped as a single piece. This further simplifies assembly and reduces weight.

In summary, the reduction of weight of the face protector with respect to conventional welding masks is achieved by a combination of at least some of the following measures:

the shell having a box-like structure;

the shape of the front section and the nose bridge section;

the lower sections of the side sections being thinner than the remaining parts of the side sections or of the shell;

the ADF unit not being realised as a detachable and replaceable cassette; and

the ADF pane and optionally also the electronics unit being held in the shell in a sealed manner by the ADF frame, without any further mounting parts and without separate casings or frames for the ADF pane and or the electronics unit.

The balance of the face protector is improved by the reduction in weight and furthermore by at least a combination of at least some of the following measures:

the shell being close to the wearer's face; and

the stiffener transferring part of the weight of the hard shell unit to the middle and to the back end of the cap.

Note: as a rule, when in the present description reference is made to “upper”, “lower”, “horizontal”, “vertical”, “front” or “back” and the like, then this is understood to relate to the situation when the face protector is worn by a user, with the user's or wearer's head being in an upright position.

When plastic materials are mentioned, this as a rule includes fibre reinforced plastic materials.

Further embodiments are evident from the dependent patent claims.

### BRIEF DESCRIPTION OF THE DRAWINGS

The subject matter of the invention will be explained in more detail in the following text with reference to exemplary embodiments which are illustrated in the attached drawings.

FIG. 1 is a perspective view of a face protector.

FIG. 2 is an exploded perspective view of main components of the face protector.

FIG. 3 is a further exploded perspective view of components of the face protector.

FIGS. 4a-4b are perspective views of a shell part of the face protector.

FIG. 5 is an elevation view of the face protector in relation to a user's head.

FIG. 6 is a rear perspective view of the face protector in relation to a user's head.

FIG. 7 is a perspective view showing an ADF frame.

FIG. 8 is a perspective view showing a protective shield.

FIG. 9 is a sectional view of the shell.

FIG. 10 is the same sectional view as FIG. 9, but with the ADF frame and protective shield attached to the shell.

FIG. 11 is a sectional view with the ADF unit arranged between the shell and the ADF frame.

FIG. 12 is a vertical sectional view with the ADF unit and its electronics unit arranged between the shell and the ADF frame.

FIG. 13 is a perspective view showing a support element in the cap.

FIG. 14 illustrates the mask's field of view relative to a user's head.

FIG. 15 is a schematic illustration showing a comparison of fields of view.

In principle, identical parts are provided with the same reference symbols in the figures.

### DETAILED DESCRIPTION OF THE INVENTION

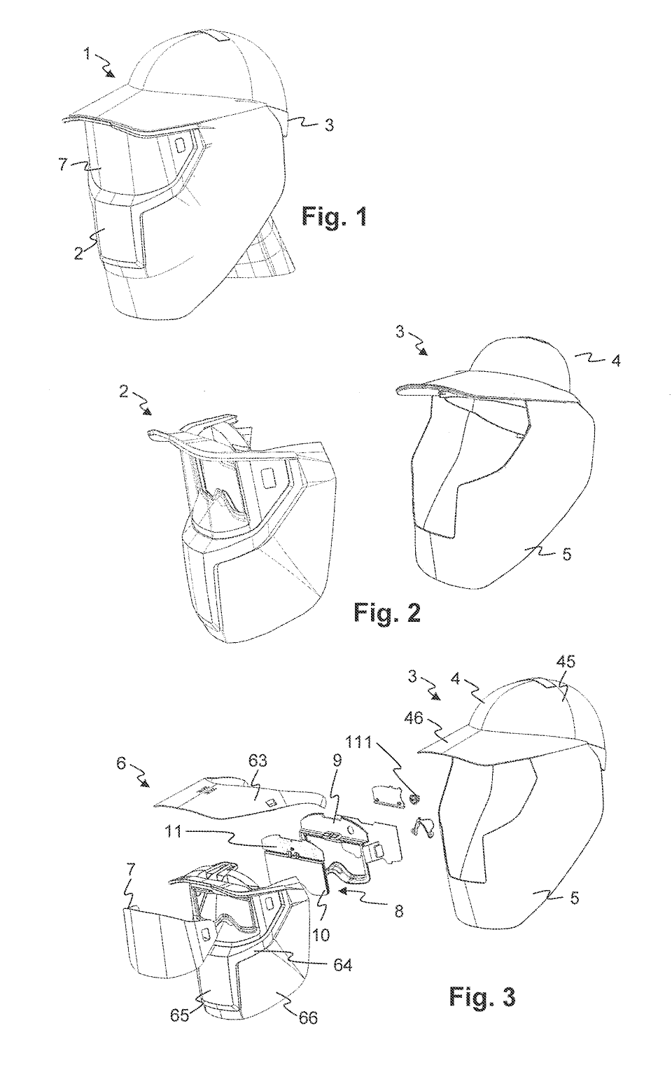

Components of a face protector according to an embodiment are explained with reference to FIGS. 1 through 4. The face protector 1 comprises a hard shell unit 2 and a soft shell unit 3. The hard shell unit 2 is made of a hard plastic material, for example from a material commonly used for welding masks, and is resistant to sparks and other influences occurring when welding. The shape of the hard shell unit 2 is adapted to cover and protect the user's face and head according to the relevant norms for welding masks. The soft shell unit 3 can be made mainly of a fabric, in particular of a functional fabric or textile material resistant to sparks, heat and other influences occurring when welding. The soft shell unit 3 comprises a cap 4 and a neck protector 5. The cap 4 can be essentially shaped like a baseball cap, that is, comprising a rounded crown 45 and a stiff visor 46 or peak projecting in front. The hard shell unit 2 and soft shell unit 3 can be attached to each other and thus can be put on by the user or taken off together.

The hard shell unit 2 comprises as main part a shell 6 carrying the other parts, in particular an Auto Darkening Filter (ADF) unit 8, an ADF frame 9 and a protective shield 7.

The ADF unit 8 in turn comprises an ADF pane 10 with the actual filter pane, and an electronics unit 11. The electronics unit 11 typically comprises a battery and/or solar cells, and one or more sensors and/or communication devices in order to detect when a welding operation or a bright light occurs that could damage or affect the user's eyes in other ways. The ADF pane 10 comprises the actual ADF, typically an electro-optical filter. The ADF pane 10 can comprise a liquid crystal shutter driven by means of the electronics unit 11 to darken the shutter when the electronics unit 11 detects a welding process occurring. The ADF pane 10 can further comprise transparent protective plates, e.g. from polycarbonate or glass etc, between which the actual ADF is sandwiched. ADF shutters in general and associated welding detection methods are known to the skilled person.

The protective shield 7 serves to protect the ADF pane 10 from sparks and other emissions of the welding process. The protective shield 7 can be easily exchanged by the user, in particular without the use of tools to remove the protective shield 7. The protective shield 7 is shaped such that a plurality of protective shields 7 can be stacked on each other, nesting with each other such as to occupy little space when stored.

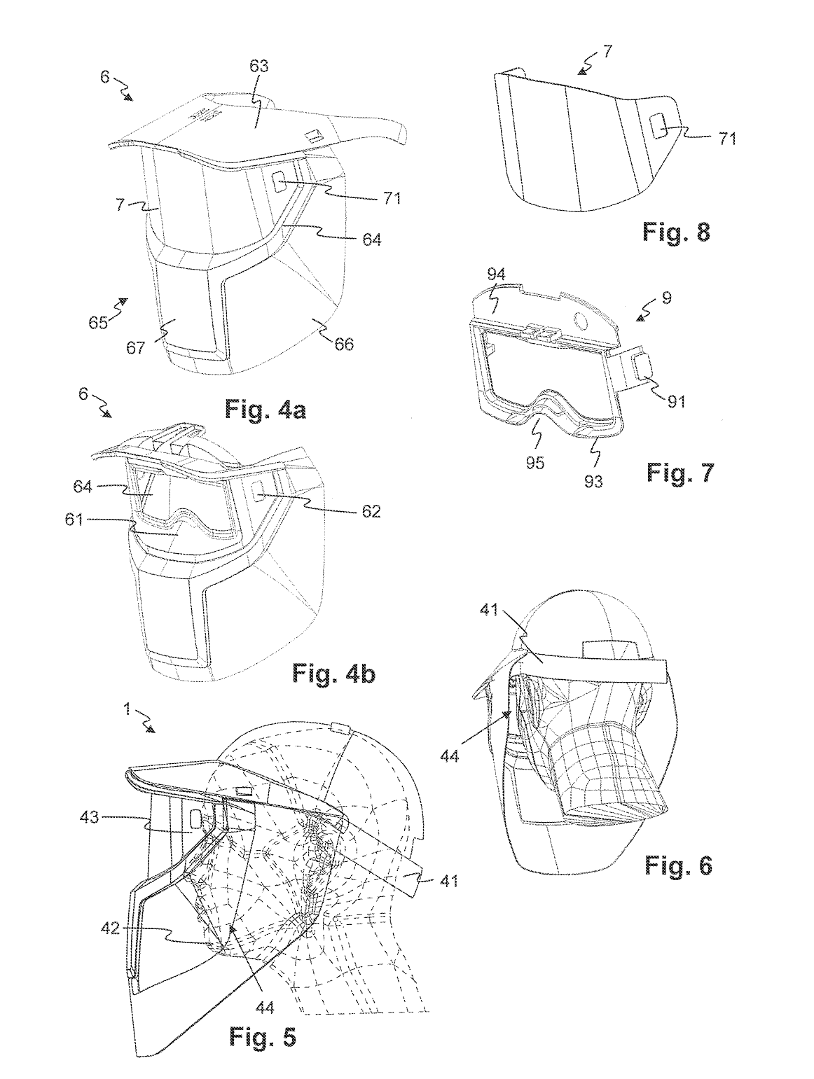

FIGS. 4a and b show a shell 6 of the face protector by itself. The shell 6 is shaped as a single piece of plastic. It comprises a frame with two essentially vertical side sections 64 joined at the top by an essentially horizontal transverse top section 63. The top section 63 is oriented essentially at a right angle to the side sections 64 and is joined to the side sections 64 along essentially horizontal edges. A front section 65 is joined to the side sections 64 along essentially vertical edges. As a result, the top, side and front sections form part of a box-like structure that gives the shell 6 structural stability. In addition, the front section 65 comprises a convex section 67 that is curved in a convex shape, and a nose bridge 61 section that is shaped to extend at an angle to the convex section 67. This further stiffens the front section 65 and thus also the entire shell 6. The front section 65 comprises a cutout or window section in which the ADF pane 10 is mounted.

The top section 63 is covered by the visor 46 and is shaped corresponding to the shape visor 46. This correspondence can be with regard to curvature of the top section 63 and the visor 46, and/or with regard to their contour. The top section 63 can comprise fastening elements for attaching the visor 46 to the top section 63. Such fastening elements also serve to transfer at least part of the force exerted by the weight of the shell 6 to the cap 4. The top section 63 can be detachable and can be part of a stiffener 47, shown in more detail in FIG. 13. FIG. 4a shows the shell 6 with the top section 63 and protective shield 7 in place, and FIG. 4b shows the shell with these parts detached.

The neck protector 5 covers or overlaps with lower sections of the front section 65 and of the side sections 64. Lower sections 66 of the side sections are covered by corresponding side sections of the neck protector 5 and serve to distance the neck protector 5 from the user's neck, providing a ventilation space 44 that helps to aerate the face protector 1. The lower sections 66 of the side section are less important for the structural stability of the shell 6 as a whole and can be made of thinner material than the other sections of the shell 6.

Typical material thickness for the shell 6 and for the lower section 66 of the side section is between 1 mm and 2 mm, and in particular is 1.2 mm.

FIG. 5 shows a side view of the face protector in relation to a user's or wearer's head. In addition to the parts already described, a head strap 41 is shown. The head strap 41 can be made of reinforced material and is arranged to take up part of the weight of the shell 6. The head strap 41 can be elastic or adjustable to the size of the wearer's head. Essentially (when the face protector 1 is worn) the remaining weight of the shell 6 is carried by the visor 46 which in turn is held by the crown 45 of the cap 4. This distributes the force exerted by the weight of the shell 6 over the user's head, making the face protector 1 comfortable to carry. In order to assist in this distribution of force, the cap 4 can comprise inserts (or stiffeners, braces, struts, reinforcements). Such stiffeners can be sewn into or attached to the cap 4 or inserted into corresponding openings provided in the cap 4.

FIG. 13 shows a schematic view of a stiffener 47 as part of the cap 4 (the shell 6 is not drawn). The stiffener 47 can be sewn into or otherwise attached to the cap 4. The stiffener 47 transfers and distributes forces acting on the visor 46 to side sections and optionally also back sections of the cap 4. The stiffener 47 typically is made of a plastic material but also can be made of metal.

The side view of FIG. 5 also shows a forehead space 43 between the visor 46, ADF unit 8 and the user's face, and a breathing space 42 between the front section 65 and the user's mouth and chin.

FIG. 6 shows a view of the face protector in relation to a user's head from below and from the back. The ventilation space 44 created by the lower section 66 of the side section holding the neck protector 5 away from the user's neck is made visible.

FIG. 7 shows an ADF frame 9. The ADF frame 9 comprises a frame section 93 that is designed to hold the ADF pane 10 against the shell 6, and a cover section 94 for covering and protecting the electronics unit 11. The frame section 93 comprises a nose cutout 95 in its lower edge, corresponding to the location of the wearer's nose. The two sections 93, 94 can be integrally shaped, that is, shaped as a single piece. This typically is done by manufacturing the ADF frame 9 from a plastic material by an injection moulding or other moulding process. The ADF frame 9 further comprises, at each side, a latch 91 which can assist in attaching the ADF frame 9 to the shell 6. The latch 91 too, can be integrally shaped with the other parts of the ADF frame 9. That is, the latch 91 can be shaped as a single piece with at least the frame section 93 and optionally also the cover section 94.

FIG. 8 shows a protective shield 7. The protective shield 7 comprises a contoured bottom edge with a shape corresponding to that of the nose bridge 61 of the shell 6. Two lateral sections of the protective shield 7 comprise shield slots 71 for releasably attaching the protective shield 7 to the shell 6.

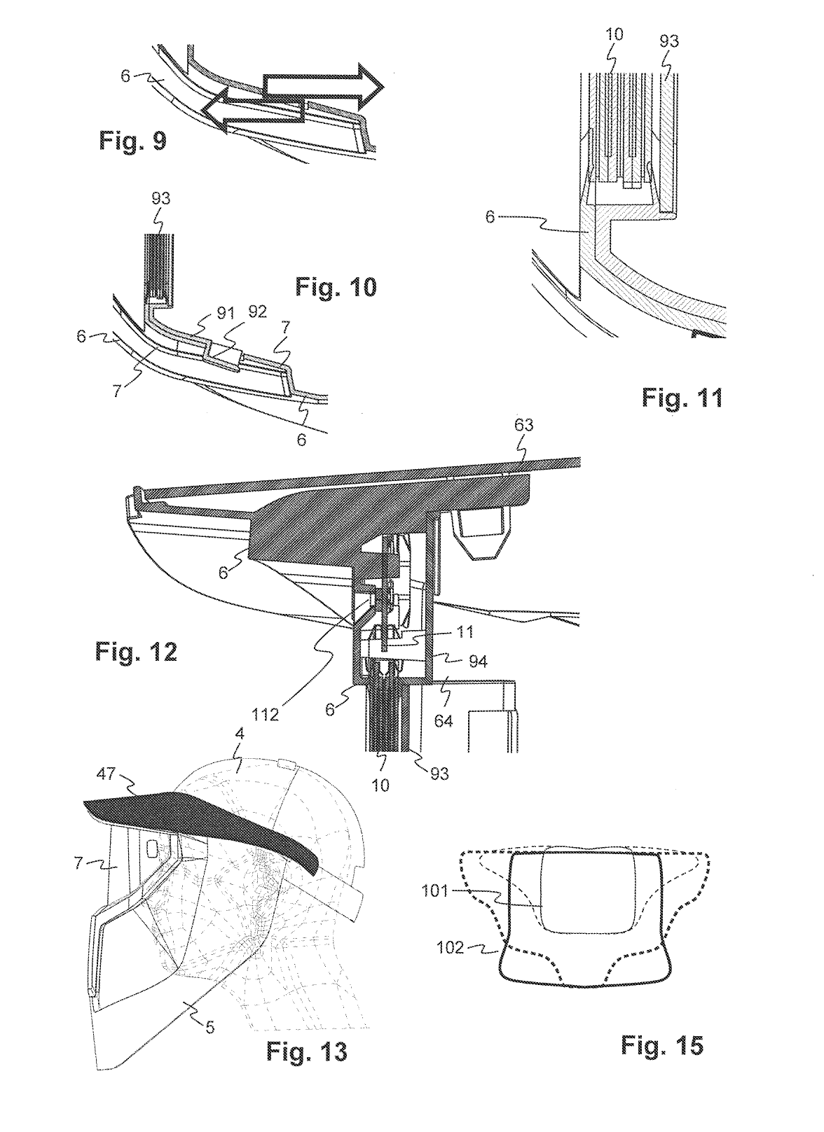

FIG. 9 shows a horizontal sectional view of the shell 6 in the region of one of the shell slots 62. Two arrows are shown, indicating the movement of forming tools that can used to form the shell 6, e.g. in an injection moulding process. There are no undercuts. The forming tools can thus be shaped without cams. Deforming, that is, separating the moulds, can be done by moving the moulds in the directions indicated by the arrows. The shafts of the arrows schematically indicate the shape of the moulds in the region of the shell slot 62.

FIG. 10 shows the same horizontal sectional view, with the ADF frame and protective shield 7 attached to the shell (for clarity, the ADF pane is omitted). The latch 91 reaches through the slot 62 of the shell 6 and then through the slot 71 of the protective shield 7, holding the protective shield 7 in place. For example, a hook 92 at an outer end of the latch 91 holds the protective shield 7 by the shield slot 71. The shell 6 and protective shield 7 can thus be manufactured from relatively stiff materials, and the latch 91 can be manufactured from a more resilient material, thereby allowing for a snap fit for attaching the protective shield 7 to the other parts of the hard shell unit 2.

FIG. 11 shows another horizontal sectional view showing the ADF unit 8 held between the shell 6 and the ADF frame 9. Around the circumference of the ADF unit 8, the shell 6 and the ADF frame 9 can be joined by gluing or welding such as, for example, ultrasonic welding or laser welding, etc. The joint can be profiled, as shown, with a groove in the ADF frame 9 and a rib on the ADF unit 8, or vice versa.

FIG. 12 shows a vertical sectional view through the upper part of the ADF unit 8 and the ADF frame 9 and a corresponding part of the shell 6. The electronics unit 11 is enclosed between the cover section 94 of the ADF frame 9 and a corresponding part of the shell 6 located above the ADF pane 10. On the inside of the mask, operating elements such as a knob 111 operatively connected to the electronics unit 11 and also arranged on the electronics unit 11 pass through the cover section 94. Towards the outer side of the mask, a sensor 112 arranged on and operatively connected to the electronics unit 11 reaches at least partially through a corresponding opening in the shell 6. This allows the sensor 112 to capture light emitted by a welding operation, for automatically activating the ADF. The cover section 94 can be welded, by laser welding or ultrasonic welding, for example, or glued, or attached in another way to the shell 6, preferably forming a gas tight joint protecting the electronics unit 11.

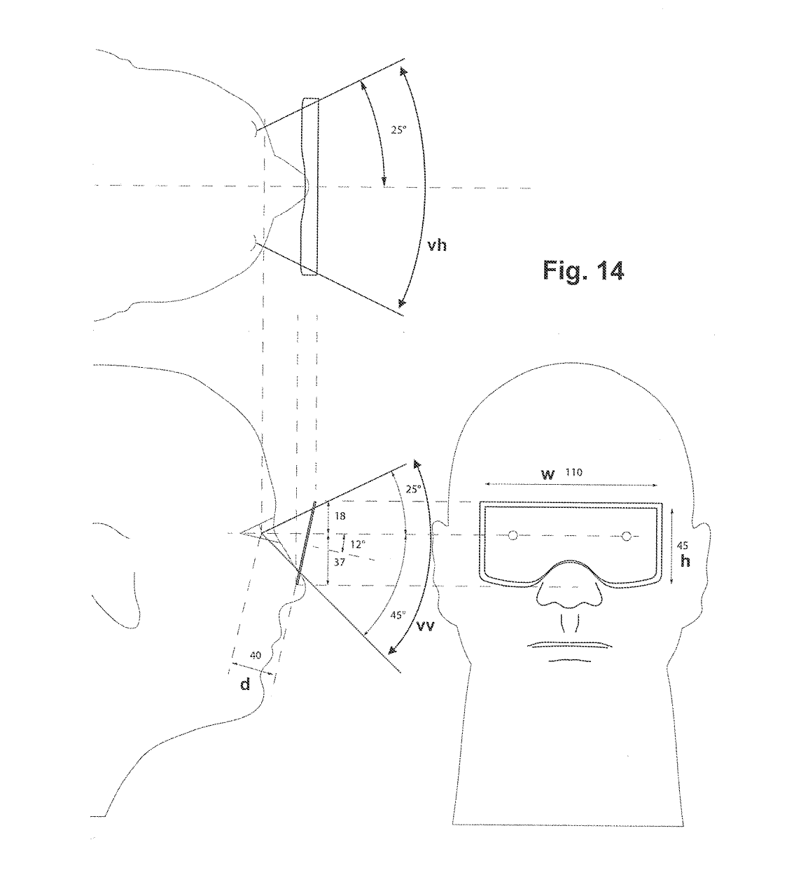

FIG. 14 shows dimensions of the ADF pane 10 and the mask's field of view relative to a user's head.

The distance from the wearer's pupils to the ADF pane 10 lies in the range of 30 mm to 50 mm, in particular in the range of 35 mm to 45 mm and more particularly at 40 mm.

The vertical extension h of the (when in the transmissive state) transparent part of the ADF pane 10 is at least 40 mm, in particular at least 45 mm.

The horizontal extension w of the transparent part of the ADF pane 10 is at least 100 mm, in particular at least 110 mm.

As a result:

a horizontal viewing angle vh for binocular viewing is at least 50°, in particular at least 60° (the viewing angle being computed for the average pupillary distance of 62 mm), and

a vertical viewing angle vv is at least 60°, in particular at least 70°.

Compared to typical conventional welding masks, this is an increase of approximately two times 17.5°, that is approximately 35° in the horizontal direction, and approximately 15° in the vertical direction.

FIG. 15 shows a comparison of fields of view for a typical welding mask, indicated by a smaller outline 101 and for a face protector as described herein, indicated by a larger outline 102. The field of view is increased by approximately 170%.

While the invention has been described in present embodiments, it is distinctly understood that the invention is not limited thereto, but may be otherwise variously embodied and practised within the scope of the claims.

1. A face protector for protecting a welder's eyes and face, comprising an auto-darkening welding filter unit that, in turn, comprises a filter pane or ADF pane and an electronics unit driving the ADF pane, the electronics unit being configured to switch the ADF pane automatically from a transparent state to a darkened state when a welding operation takes place,
the face protector further comprising a hard shell unit and a soft shell unit attached to each other,
the hard shell unit comprising a shell carrying an ADF unit with the ADF and being shaped to protect a wearer's face, the shell being manufactured of a plastic material;
the soft shell unit comprising a cap for protecting the crown of the wearer's head and a neck protector for protecting the wearer's neck, the soft shell unit being made of a combination of flexible fabric with support elements.

2. The face protector of claim 1, wherein the face protector is configured such that when it is worn by a wearer, a distance from the wearer's pupils to the ADF pane lies in the range of 30 mm to 50 mm, in particular in the range of 35 mm to 45 mm.

3. The face protector of claim 1, wherein a vertical extension of the transparent part of the ADF pane is at least 40 mm, in particular at least 45 mm.

4. The face protector of claim 1, wherein a horizontal extension of the transparent part of the ADF pane is at least 100 mm, in particular at least 110 mm.

5. The face protector of claim 1, wherein a lower edge of the ADF pane comprises a concave cutout for accommodating the wearer's nose.

6. The face protector of claim 1, wherein the ADF pane is curved, with side portions of the ADF pane, relative to a central portion of the ADF pane, curving backwards.

7. The face protector of claim 1, wherein the hard shell unit comprises a protective shield, the protective shield being attached by a releasable connection, the protective shield optionally being shaped such that a plurality of protective shields can be stacked on each other, nesting with each other.

8. The face protector of claim 1, wherein the cap is shaped in the manner of a baseball cap, comprising a rounded crown and a stiff visor projecting in front, the visor covering a transverse top section of the shell, the top section protecting a forehead space enclosed by the hard shell unit, and the visor supporting at least some of the weight of the hard shell unit when the face protector is worn by a wearer.

9. The face protector of claim 8, wherein the cap comprises a stiffener sewn in or attached to the other parts of the cap, the stiffener holding up the visor against at least part of the weight of the hard shell unit.

10. The face protector of claim 1, wherein a top section, two side sections and a front section of the shell form a box-like structure that gives the shell structural stability, and wherein the front section comprises a convex section that is curved in a convex shape, and a nose bridge section that is shaped to extend at an angle to the convex section, further stiffening the front section.

11. The face protector of claim 1, wherein the shell comprises lateral side sections, a lower section of each side section being shaped to keep the neck protector distanced from the wearer's neck, thereby establishing a ventilation space between the neck protector and the wearer's neck.

12. The face protector of claim 11, wherein the lower sections of the side sections are made of thinner material than the remaining sections of the side sections, and optionally also thinner than the other sections of the shell.

13. The face protector of claim 1, wherein the ADF pane is mounted on the shell via a frame section of the ADF frame, the frame section being attached to, in particular glued or welded to, the shell and holding the ADF pane in position between the frame section and the shell.

14. The face protector of claim 13, wherein the electronics unit is mounted on the shell via a cover section of the ADF frame, the cover section being attached to, in particular glued or welded to, the shell and holding the electronics unit in position between the cover section and the shell.

15. The face of claim 14, wherein the frame section and the cover section are integrally shaped as a single piece.

16. The face protector of claim 13, wherein the ADF frame comprises at least one latch, the ADF frame is mounted at the inside of the shell with the latch reaching through a slot of the shell and forming a snap fit with the protective shield, holding the protective shield in place on the outer side of the shell.

17. The face protector of claim 16, wherein at least the frame section and the latch are integrally shaped as a single piece.

18. The face protector of claim 14, wherein the ADF frame comprises at least one latch, the ADF frame is mounted at the inside of the shell with the latch reaching through a slot of the shell and forming a snap fit with the protective shield, holding the protective shield in place on the outer side of the shell.

19. The face protector of claim 18, wherein at least the frame section and the latch are integrally shaped as a single piece.

20. The face protector of claim 15, wherein the ADF frame comprises at least one latch, the ADF frame is mounted at the inside of the shell with the latch reaching through a slot of the shell and forming a snap fit with the protective shield, holding the protective shield in place on the outer side of the shell.
